(19) United States
(12) Patent Application Publication
(10) Pub. No.: US 2014/0371503 A1
(43) Pub. Date: Dec. 18, 2014

(54) SINGLE STAGE REACTOR SYSTEM WITH OXIDATIVE PREHEAT FOR DEHYDROGENATION OF HYDROCARBONS

(71) Applicant: UOP LLC, Des Plaines, IL (US)

(72) Inventors: Daniel H. Wei, Naperville, IL (US); Laurence O. Stine, Western Springs, IL (US)

(21) Appl. No.: 13/920,311

(22) Filed: Jun. 18, 2013

(51) Int. Cl. C07C 5/48

(52) U.S. Cl. CPC C07C 5/48
USPC 585/658; 585/300

(57) ABSTRACT

A single stage dehydrogenation reactor system including a charge heater and one or more reactors is described. The hydrocarbon feed is combined with hydrogen and heated in a charge heater to a temperature lower than the dehydrogenation temperature to avoid thermal cracking. Before entering the dehydrogenation reactors, oxygen is added. The oxidative preheat then takes place in the presence of the dual functional catalyst which has dehydrogenation and selective oxidation activities. The oxygen selectively burns hydrogen and raises the reaction temperature, and the dehydrogenation reaction then occurs.

### BACKGROUND OF THE INVENTION

Catalytic dehydrogenation can be used to convert paraffins to the corresponding olefin, e.g., propane to propene, or butane to butene.

FIG. 1 shows one typical arrangement for a moving bed dehydrogenation process 5. The process 5 includes a reactor section 10, a regeneration section 15, and a product recovery section 20.

The reactor section 10 includes one or more reactors 25 (four as shown). The feed 30 is sent to a heat exchanger 35 where it exchanges heat with the reactor effluent 40 to raise the feed temperature. The feed 30 is sent to a preheater 45 where it is heated to the desired inlet temperature. The preheated feed 50 is sent from the preheater 45 to the first reactor 25. Because the dehydrogenation reaction is endothermic, the temperature of the effluent 55 from the first reactor 25 is less than the temperature of the preheated feed 50. The effluent 55 is sent to interstage heaters 60 to raise the temperature to the desired inlet temperature for the next reactor 25.

After the last reactor (in this case the fourth reactor), the effluent 40 is sent to heat exchanger 35, and heat is exchanged with the feed 30. The effluent 40 is then sent to the product recovery section 20.

The catalyst 65 moves through the series of reactors 25. When the catalyst 70 leaves the last reactor 25, it is sent to the regeneration section 15. The regeneration section includes a reactor 75 where the coke on the catalyst is burned off and the catalyst may go through a reconditioning step. The regenerated catalyst 80 is sent back to the first reactor 25.

In the product recovery section 20, the effluent 40 is cooled, compressed, dried, and separated in separator 85. The gas 90 is expanded in expander 95 and then separated into a recycle hydrogen stream 100 and a net separator gas stream 105. The liquid stream 110, which includes the olefin product and unconverted paraffin, is sent for further processing, where the desired olefin product is recovered and the unconverted paraffin is recycled to the dehydrogenation reactor.

FIG. 2 shows a typical arrangement for a cyclic bed dehydrogenation process 115. The process 115 includes a reactor section 120, and a product recovery section similar to that described above (not shown in FIG. 2).

In this process 115, the feed 130 is sent to a heat exchanger 135 where it exchanges heat with the reactor effluent 140 to raise the feed temperature. As shown, there are four reactors 145A-D. Of these, typically one will be operating (145A); one will be purging (145B); one will be regenerating the catalyst, that is, burning of coke and reconditioning if required (145C); and one will be purging and preparing for the next process cycle (145D). The feed 130 is sent to preheaters 150 where it is heated to the desired inlet temperature. The preheated feed 155 is sent from the preheater 150 to the operating reactor 145A.

The effluent 140 from the operating reactor 145A is sent to heat exchanger 135, and heat is exchanged with the feed 130. The effluent 140 is then sent to the product recovery section.

Reactor 145B is being purged. The hydrocarbon feed to the reactor is stopped, and the connection to the effluent is closed. A purge gas 160 is introduced into reactor 145B to remove any hydrocarbon feed from the reactor in preparation for regenerating the catalyst.

Reactor 145C is being regenerated. An oxygen-containing stream 165 is introduced into the reactor so the coke on the catalyst can be burned off, and the catalyst is reconditioned if required.

Reactor 145D is being purged. The oxygen-containing feed to the reactor is stopped. A purge gas 160 is introduced into reactor 145D to remove any residual air/oxygen feed from the reactor in preparation for next processing cycle.

The time duration of steps two, three and four, that is purging, coke burning, and purging is matched with the time duration of the first step, that is the process cycle. In some instances, to match this timing duration, one may use more than one reactor in the processing step.

In paraffin dehydrogenation processes, maximum conversion is limited by equilibrium at the reactor outlet conditions. Feed has to be heated to a high temperature before being fed to a series of adiabatic reactors where dehydrogenation takes place. Depending on the carbon number of the feed being dehydrogenated, this temperature can vary from about 450° C. to about 700° C. The lower carbon number feeds, such as ethane, propane, butane (C₂-C₄), require higher temperatures, in the range of about 600 to about 700° C., compared to those with carbon number, such as decane or dodecane (C₁₀, C₁₂), which may require temperatures in the range of about 450 to about 550° C. As shown in FIG. 3, at 101 kPa (1 atm) and 550° C., the propylene to propane ratio is 32/68, while at the same temperature, the isobutene to isobutane ratio is 50/50. At the same reactor pressure and temperature, equilibrium conversion is higher at lower partial pressure of alkanes. This can be accomplished by adding a diluent to the reaction mixture.

The paraffin dehydrogenation reaction is equilibrium limited.

CₙH₂ₙ₊₂ [symbol] CₙH₂ₙ+H₂

As shown, the dehydrogenation reaction produces alkenes and hydrogen. Because the reaction is endothermic, the reactor outlet temperature is lower than the inlet temperature. As the temperature declines, so does the equilibrium concentration for alkene, and hence it limits the maximum conversion that can be achieved within each reactor. Furthermore, higher inlet temperature can thermally crack the feed hydrocarbons, resulting in selectivity loss.

Multi-stage heating steps increases the circuit pressure drop and hot residence time much more than the required amount for actual hydrocarbon-catalyst contact. The extended hot temperature residence promotes thermal cracking of the feed in heater tubes and transfer lines between the heaters and reactors, resulting in low selectivity. It also results in higher utilities consumption. Limited conversion increases the amount of recycled unreacted material, resulting in increases in unit capital costs and operating costs.

There is a need for improved dehydrogenation processes.

### SUMMARY OF THE INVENTION

One aspect of the invention is a process for dehydrogenation of hydrocarbons. In one embodiment, the process includes preheating a feed comprising a hydrocarbon feed, a diluent and hydrogen to a temperature lower than a dehydrogenation temperature. An oxygen-containing gas is introduced to the preheated feed. The preheated feed with the oxygen-containing gas is introduced into a dehydrogenation reaction zone comprising a dehydrogenation reactor containing a dual functional catalyst having dehydrogenation and selective oxidation activities. The preheated feed is contacted with the catalyst in the dehydrogenation reactor to selectively oxidize the hydrogen to further heat the preheated feed. The heated feed is contacted with the catalyst in the dehydrogenation reactor under dehydrogenation conditions to form olefins and hydrogen.

### BRIEF DESCRIPTION OF THE DRAWINGS

FIG. 1 is an illustration of one embodiment of a prior art dehydrogenation process.

FIG. 2 is an illustration of one embodiment of a prior art cyclic bed dehydrogenation process.

FIG. 3 is a graph showing the propylene and isobutylene equilibrium at 101 kPa (1 atm) with no hydrogen recycle.

FIG. 4 is an illustration of one embodiment of a single-stage oxidative dehydrogenation process.

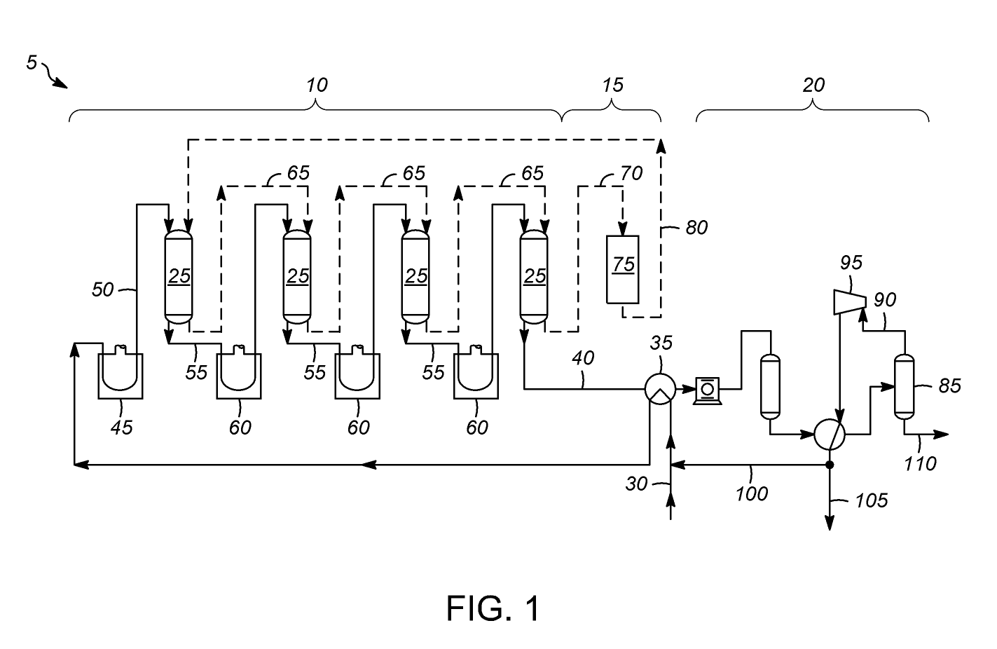

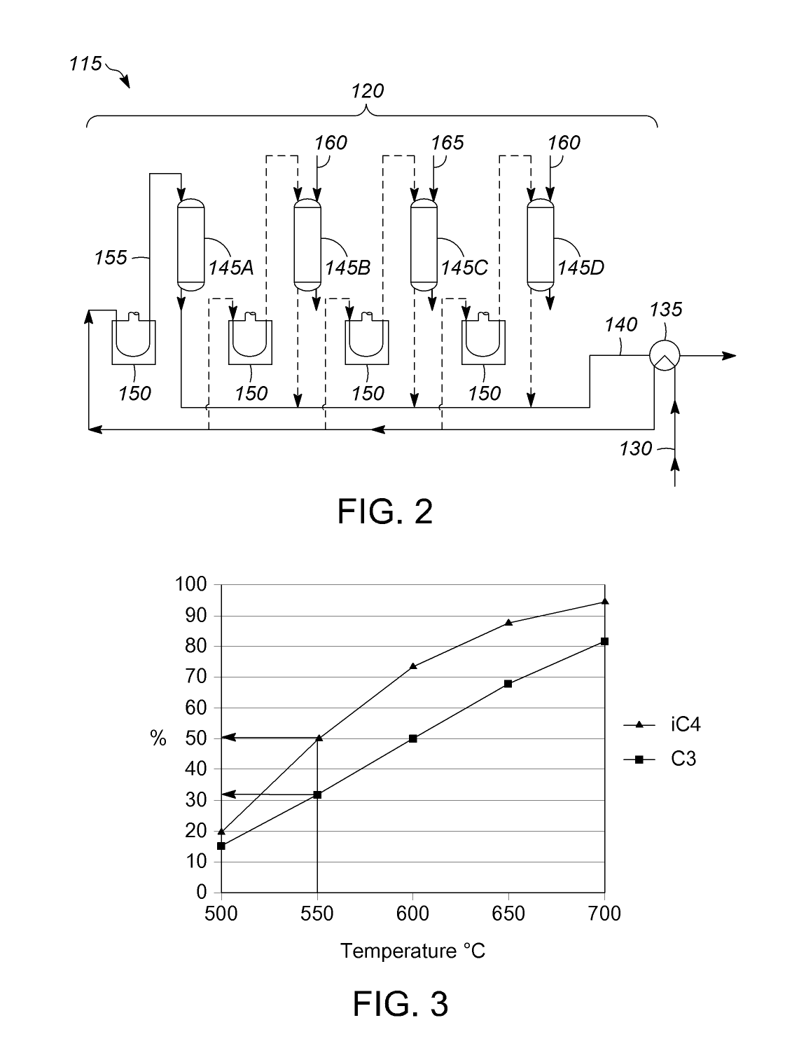

### DETAILED DESCRIPTION OF THE INVENTION

The invention involves a single stage oxidative dehydrogenation reactor system including a charge heater and one or more dehydrogenation reactors. If there is more than one reactor, the reactors are arranged in parallel. The number of reactors in parallel is determined by the hydrocarbon processing capacity.

Part of the hydrogen mixed with the hydrocarbon feed is selectively oxidized in the initial part of the reactor, oxidative preheat zone, in the presence of a dual functional catalyst designed for selective oxidation of hydrogen and alkane dehydrogenation. The reaction mixture is further heated in the oxidative preheat zone and reaches desired dehydrogenation temperature. The diluent mixed in the hydrocarbon feed shifts the equilibrium in the desired direction and brings sufficient heat to the dehydrogenation zone for achieving target alkane conversion in a single stage reactor system. Thermal cracking is, therefore, minimized by operating the charge heater at a low temperature and eliminating the need for interstage heaters

The hydrocarbon feed is combined with hydrogen and a diluent, such as steam, and then sent to the charge heater which is operated at a temperature lower than the dehydrogenation temperature to avoid thermal cracking. The temperature is high enough that the hydrogen will selectively oxidize in the presence of the catalyst, but not too high so that oxidation of hydrocarbons and hydrogen will not take place outside the reaction zone.

Before entering the reactors, oxygen and a diluent, such as steam, are added in such a way that the composition of the reaction mixture is outside the explosive envelope. The oxidative preheat then takes place in the presence of the catalyst having selective oxidation and dehydrogenation functions. Initially, the oxygen selectively burns hydrogen and raises the reaction temperature; the dehydrogenation reaction takes place when the temperature has been raised sufficiently. The steam is added in such a way that the reaction mixture in the oxidative preheating zone carries sufficient amount of heat to the dehydrogenation zone and reduces the partial pressure of the alkane to achieve the desired conversion in a single-stage reactor system. As a result of eliminating multi-stage heating steps, selectivity to alkene is higher at the same conversion level. For example, the selectivity to propylene with oxidative preheating in a single reactor system is at least about 5 wt % higher than the selectivity shown in a multi-stage heating reactor system.

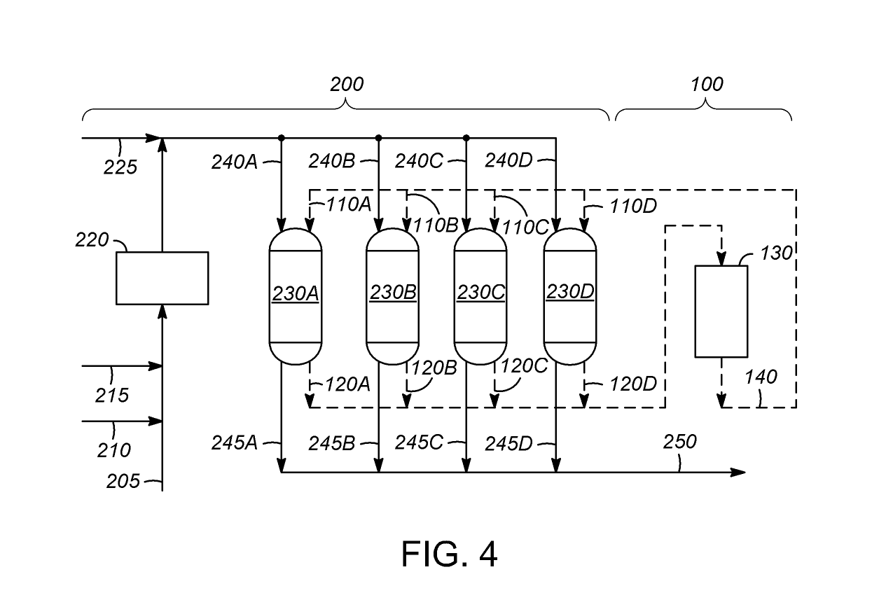

FIG. 4 is an illustration of a single stage reactor system 200 having four reactors arranged in parallel. The hydrocarbon feed 205 is mixed with a diluent 210. The hydrocarbon feed 205 with diluent 210 is mixed with hydrogen 215.

The mixture is sent to charge heater 220 where it is preheated to a temperature lower than the dehydrogenation temperature of the hydrocarbon.

An oxygen-containing gas 225 is then mixed with the preheated feed mixture which is then sent to the dehydrogenation reaction zone. The amount of oxygen-containing gas added is controlled so that it is outside the explosive region.

The dehydrogenation zone includes one or more dehydrogenation reactors 230A-D (four are shown). If there is more than one reactor, the reactors are connected in parallel.

The preheated feed with the oxygen-containing gas is split into multiple streams 240A-D to feed the dehydrogenation reactors 230A-D.

The dehydrogenation reactors 230A-D contain a catalyst having selective oxidation and dehydrogenation functions. The catalyst selectively oxidizes the hydrogen in the feed with the oxygen in the oxygen-containing gas to generate heat in the initial part of the reactor, the oxidative preheat zone. After the reaction mixture reaches the desired dehydrogenation temperature, the catalyst catalyzes the dehydrogenation of the hydrocarbon.

Before the preheated feed with the oxygen-containing gas (which contains the hydrocarbon feed, hydrogen, oxygen-containing gas, and one or more optional diluents) enters the reaction zone, the temperature is low so that oxidation and dehydrogenation reactions do not occur. However, the temperature is sufficient for the oxidation of hydrogen when the reaction mixture enters the reaction zone in the presence of the catalyst. Therefore, the hydrogen in the feed is selectively burned, raising the temperature of the hydrocarbon feed. When the temperature of the hydrocarbon feed has been raised to a desired dehydrogenation temperature, the hydrocarbon feed is dehydrogenated.

The effluent streams 245A-D from the dehydrogenation reactors 230A-D containing the olefins formed by dehydrogenation are mixed together to form a stream 250 and sent for further processing to recover the olefin product (not shown).

The catalyst 110A-D moves through the reactors 230A-D. When the catalyst 120A-D leaves the reactors 230A-D, it is sent to the regeneration section 100. The regeneration section includes a reactor 130 where the coke on the catalyst is burned off and the catalyst may go through a reconditioning step. The regenerated catalyst 140 is sent back to the reactors 230A-D.

Any dehydrogenatable hydrocarbon may be utilized as feed. Typically, the hydrocarbons which may be dehydrogenated include dehydrogenatable hydrocarbons having from 2 to 20 or more carbon atoms including paraffins, alkylaromatics, naphthenes, and olefins. One group of hydrocarbons which can be dehydrogenated is the group of paraffins having from 2 to 20 or more carbon atoms. The process is useful for dehydrogenating paraffins having from 2 to 15 or more carbon atoms to the corresponding monoolefins, or for dehydrogenating monoolefins having from 2 to 15 or more carbon atoms to the corresponding diolefins or acetylene derivatives.

The diluents added before and after the charge heater can be same or different. The diluent reduces the partial pressure of the hydrocarbon feed which shifts the equilibrium in the desired direction, and increases heat capacity of the reaction mixture for the dehydrogenation reactions. Suitable diluents include, but are not limited to, steam, methane, nitrogen, carbon dioxide, or an inert gas, or mixtures thereof. Superheated steam can help to maintain process efficiency by reducing catalyst coking tendencies.

A mixture of the hydrocarbon feed, hydrogen, and optional diluents is preheated in the charge heater to a temperature lower than the dehydrogenation temperature of the hydrocarbon. It will be higher than the oxidation temperature of hydrogen in the presence of the catalyst but lower than the oxidation temperature of hydrogen without the catalyst. The temperature will typically be in the range of about 480° C. to about 580° C., or about 500° C. to about 570° C. The preheat temperature will depend on the particular hydrocarbon being dehydrogenated.

Any oxygen containing gas can be added after the charge heater. Suitable oxygen-containing gases include, but are not limited to, air, pure oxygen, and an oxygen stream containing more oxygen than air.

When the preheated feed with the oxygen-containing gas is introduced into the dehydrogenation zone containing the dual functional catalyst, the temperature is lower than the dehydrogenation temperature, but higher than the oxidation temperature for hydrogen. Therefore, the hydrogen is oxidized, raising the temperature of the hydrocarbon feed to a desired dehydrogenation temperature, typically about 500° C. to about 900° C., or 500° C. to about 800° C., or about 500° C. to about 700° C., or about 510° C. to about 700° C., or about 520° C. to about 700° C., or about 530° C. to about 700° C., or about 540° C. to about 700° C., or about 550° C. to about 700° C., or about 560° C. to about 700° C., or about 570° C. to about 700° C., or about 580° C. to about 700° C., or about 590° C. to about 700° C., or about 600° C. to about 700° C.

The exact dehydrogenation conditions are a function of the particular dehydrogenatable hydrocarbon undergoing dehydrogenation. Such conditions include a reaction pressure in the range of from about 0.01 to about 4.1 MPa (0.1 to about 40 atm), or from about 0.1 to about 2.0 MPa (1 to 20 atm). Other reaction conditions will include a liquid hourly space velocity based on the total hydrocarbon charge rate of from about 0.1 to about 100 hr⁻¹, steam-to-hydrocarbon molar ratios ranging from about 0.1:1 to about 40:1, and hydrogen to hydrocarbon molar ratio ranging from 0.1:1 to about 10:1.

The contacting step may be accomplished by using the catalyst in a fixed bed system, a moving bed system, a fluidized bed system, or in a batch type operation. However, in view of the fact that the attrition losses of the valuable catalyst should be minimized and of the well known operational advantages, it is desirable to use either a fixed bed catalytic system, or a dense phase moving bed system such as is shown in U.S. Pat. No. 3,725,249.

If a fixed bed catalytic reaction system is used, it is anticipated that the reaction system could take many forms. The first possibility is that the reaction would comprise a single reaction zone within one or more reactor arranged in parallel, each reactor with single inlet and outlet ports. The feed hydrocarbon, steam, and any and all co-feeds would enter the inlet of the reactor and products and by-products would leave the system through the reactor outlet port. It is, of course, understood that the dehydrogenation reaction zone may be two or more distinct catalyst containing zones. The reactants may be contacted with the catalyst bed in either upward, downward, or radial flow fashion with the latter being preferred. In addition, the reactants may be in the liquid phase, admixed liquid-vapor phase, or a vapor phase when they contact the catalyst, with the best results obtained in the vapor phase. The dehydrogenation reaction system then preferably comprises a dehydrogenation reaction step containing one or more fixed or dense-phase moving beds of the above-described catalytic composite.

The effluent stream from the dehydrogenation zone generally will contain unconverted dehydrogenatable hydrocarbons, hydrogen and the products of dehydrogenation reactions. This effluent stream is typically cooled and passed to a hydrogen separation step to separate a hydrogen-rich vapor phase from a hydrocarbon-rich liquid phase. Generally, the hydrocarbon-rich liquid phase is further separated using a suitable selective adsorbent, a selective solvent, a selective reaction or reactions, or a suitable fractionation scheme. Unconverted dehydrogenatable hydrocarbons are recovered and may be recycled to the dehydrogenation step. Products of the dehydrogenation reactions are recovered as final products or as intermediate products in the preparation of other compounds.

It is an aspect of this invention that dehydrogenation conversion process be a complete process. That is to say, the process will comprise a reaction section and other sections such as gas recycle, liquid recycle, product recovery, and the like such that the process is viable and efficient. Examples of some of the product recovery techniques that could be employed alone or in combination in the product recovery zone of a hydrocarbon conversion process are: distillation including vacuum, atmospheric, and superatmospheric distillation; extraction techniques including, for example, liquid/liquid extractions, vapor/liquid extractions, supercritical extractions and other; absorption techniques, adsorption techniques, and any other known mass transfer techniques which can achieve the recovery of the desired products.

The catalyst is an oxidative dehydrogenation catalyst. This dual function oxidative dehydrogenation catalyst enables dehydrogenation of the hydrocarbon feed and also promotes selective oxidation of hydrogen with added oxygen forming water. The oxidative dehydrogenation catalyst does not supply oxygen for the reaction, that is, the catalyst is not in its oxide form. Rather, the oxygen for the reaction is added to the reactors.

The oxidative dehydrogenation catalyst generally comprises a first component selected from the group consisting of Group VIII metal components and mixtures thereof on a support.

In some embodiments, the oxidative dehydrogenation catalyst includes a second component selected from the group consisting of alkali metal components, alkaline earth metal components, and mixtures thereof.

In some embodiments, the oxidative dehydrogenation catalyst includes a third component selected from the group consisting of tin, germanium, lead, indium, gallium, thallium, and mixtures thereof.

As indicated above, the catalyst includes a first component selected from Group VIII metals or mixtures thereof, with Group VIII noble metals being preferred. The Group VIII noble metal may be selected from the group consisting of platinum, palladium, iridium, rhodium, osmium, ruthenium, or mixtures thereof, with platinum being preferred.

The Group VIII metal component is desirably well dispersed throughout the catalyst. It generally will comprise about 0.01 to 5 wt. %, calculated on an elemental basis, of the final catalytic composite. Preferably, the catalyst comprises about 0.1 to 2.0 wt. % Group VIII metal component, especially about 0.1 to about 2.0 wt. % platinum.

The Group VIII metal component may be incorporated in the catalyst in any suitable manner such as, for example, by coprecipitation or cogelation, ion exchange or impregnation, or deposition from a vapor phase or from an atomic source, or by like procedures either before, while, or after other catalytic components are incorporated. The preferred method of incorporating the Group VIII metal component is to impregnate the support with a solution or suspension of a decomposable compound of a Group VIII metal. For example, platinum may be added to the support by commingling the latter with an aqueous solution of chloroplatinic acid. Another acid, for example, nitric acid or other optional components, may be added to the impregnating solution to further assist in evenly dispersing or fixing the Group VIII metal component in the final catalyst.

The catalyst can also include a second catalytic component comprised of an alkali or alkaline earth component. The alkali or alkaline earth component may be selected from the group consisting of cesium, rubidium, potassium, sodium, and lithium or from the group consisting of barium, strontium, calcium, and magnesium or mixtures of metals from either or both of these groups. It is believed that the alkali and alkaline earth component exists in the final catalyst in an oxidation state above that of the elemental metal. The alkali and alkaline earth component may be present as a compound such as the oxide, for example, or combined with the support or with the other catalytic components.

Preferably the alkali and alkaline earth component is well dispersed throughout the catalytic composite. The alkali or alkaline earth component will preferably comprise 0.9 to 1.1 wt. %, calculated on an elemental basis of the final catalytic composite.

The alkali or alkaline earth component may be incorporated in the catalytic composite in any suitable manner such as, for example, by coprecipitation or cogelation, by ion exchange or impregnation, or by like procedures either before, while, or after other catalytic components are incorporated. A preferred method of incorporating the alkali component is to impregnate the support with a solution of potassium hydroxide.

The catalyst can also include a modifier metal component comprising Group IIIA or IVA metals. The modifier metal component can be selected from the group consisting of tin, germanium, lead, indium, gallium, thallium, and mixtures thereof. The effective amount of the third modifier metal component is preferably uniformly impregnated. Generally, the catalyst will comprise from about 0.01 to about 10 wt. % of the third modifier metal component calculated on an elemental basis on the weight of the final composite. Preferably, the catalyst will comprise from about 0.1 to about 5 wt. % of the third modifier metal component.

The third modifier metal component is preferably tin. Some or all of the tin component may be present in the catalyst in an oxidation state above that of the elemental metal. This component may exist within the composite as a compound such as the oxide, sulfide, halide, oxychloride, aluminate, etc., or in combination with the support or other ingredients of the composite. Preferably, the tin component is used in an amount sufficient to result in the final catalytic composite containing, on an elemental basis, about 0.01 to about 10 wt. % tin, with best results typically obtained with about 0.1 to about 5 wt. % tin.

Suitable tin salts or water-soluble compounds of tin which may be used include stannous bromide, stannous chloride, stannic chloride, stannic chloride pentahydrate, stannic chloride tetrahydrate, stannic chloride trihydrate, stannic chloride diamine, stannic trichloride bromide, stannic chromate, stannous fluoride, stannic fluoride, stannic iodide, stannic sulfate, stannic tartrate, and the like compounds. The utilization of a tin chloride compound, such as stannous or stannic chloride is particularly preferred.

The third component of the catalyst may be composited with the support in any sequence. Thus, the first or second component may be impregnated on the support followed by sequential surface or uniform impregnation of the third component. Alternatively, the third component may be surface or uniformly impregnated on the support followed by impregnation of the other catalytic components.

The catalyst may also contain a halogen component. The halogen component may be fluorine, chlorine, bromine, or iodine, or mixtures thereof. Chlorine is the preferred halogen component. The halogen component is generally present in a combined state with the support and alkali component. Preferably, the halogen component is well dispersed throughout the catalytic composite. The halogen component may comprise from more than 0.01 wt. % to about 15 wt. %, calculated on an elemental basis, of the final catalyst.

The halogen component may be incorporated in the catalyst in any suitable manner, either during the preparation of the carrier material or before, while, or after other catalyst components are incorporated. For example, the alumina sol utilized to form an alumina support may contain halogen and thus contribute at least some portion of the halogen content in the final catalyst composite. Also, the halogen component or a portion thereof may be added to the catalyst composite during the incorporation of the support with other catalyst components, for example, by using chloroplatinic acid to impregnate the platinum component. Also, the halogen component or a portion thereof may be added to the catalyst composite by contacting the catalyst with the halogen or a compound or solution containing the halogen before or after other catalyst components are incorporated with the carrier material. Suitable compounds containing the halogen include acids containing the halogen, for example, hydrochloric acid. Alternatively, the halogen component or a portion thereof may be incorporated by contacting the catalyst with a compound or solution containing the halogen in a subsequent catalyst regeneration step. In the regeneration step, carbon deposited on the catalyst as coke during use of the catalyst in a hydrocarbon conversion process is burned off, and the catalyst and the platinum group component on the catalyst are redistributed to provide a regenerated catalyst with performance characteristics much like the fresh catalyst. The halogen component may be added during the carbon burn step or during the platinum group component redistribution step, for example, by contacting the catalyst with a hydrogen chloride gas. Also, the halogen component may be added to the catalyst composite by adding the halogen or a compound or solution containing the halogen, such as propylene dichloride, for example, to the hydrocarbon feed stream or to the recycle gas during operation of the hydrocarbon conversion process. The halogen may also be added as chlorine gas (Cl₂).

The support can be a porous, absorptive support. It will typically have a surface area of from about 25 to about 500 m²/g. The support should be relatively refractory to the conditions utilized in the hydrocarbon conversion process. Suitable supports are those which have traditionally been utilized in hydrocarbon conversion catalysts such as, for example; (1) activated carbon, coke, or charcoal; (2) silica or silica gel, silicon carbide, clays, and silicates, including synthetically prepared and naturally occurring ones, which may or may not be acid treated, for example, attapulgus clay, china clay, diatomaceous earth, fuller's earth, kaolin, kieselguhr, etc.; (3) ceramics, procelain, crushed firebrick, bauxite; (4) refractory inorganic oxides such as alumina, titanium dioxide, zirconium dioxide, chromium oxide, beryllium oxide, vanadium oxide, cerium oxide, hafnium oxide, zinc oxide, magnesia, boria, thoria, silica-alumina, silica-magnesia, chromia-alumina, alumina-boria, silica-zirconia, etc.; (5) crystalline zeolitic aluminosilicates such as naturally occurring or synthetically prepared mordenite and/or faujasite, for example, either in the hydrogen form or in a form which has been exchanged with metal cations; (6) spinels such as MgAl₂O₄, FeAl₂O₄, ZnAl₂O₄, CaAl₂O₄, and other like compounds having the formula MO-Al₂O₃ where M is a metal having a valence of 2; and (7) combinations of materials from one or more of these groups. In some embodiments, the support is alumina, especially gamma-, eta-, or theta-alumina.

In some embodiments, the support is alumina having a surface area greater than about 50 m²/g, or less than 120 m²/g, or about 50 m²/g to about 120 m²/g. In addition, in some embodiments, the alumina can have an apparent bulk density (ABD) of about 0.5 g/cm³ or more, or about 0.6 g/cm³ or more. The alumina support may be prepared in any suitable manner from synthetic or naturally occurring raw materials. The support may be formed in any desired shape such as spheres, pills, cakes, extrudates, powders, granules, etc. A preferred shape of alumina is the sphere.

To make alumina spheres, aluminum metal is converted into an alumina sol by reacting it with a suitable peptizing agent and water, and then dropping a mixture of the sol into an oil bath to form spherical particles of the alumina gel. The third modifier metal component may be added to the alumina sol before it is reacted with the peptizing agent and dropped into the hot oil bath. Other shapes of the alumina carrier material may also be prepared by conventional methods. After the alumina particles optionally containing the co-formed third component are shaped, they are dried and calcined.

The drying and calcination of the alumina base component helps to impart the catalyst base with the desired characteristics. Calcination temperatures ranging from 800° C. to 950° C. are known to produce alumina comprising essentially crystallites of gamma-alumina. Calcination temperatures of 1100° C. and above are known to promote the formation of alpha-alumina crystallites while temperatures of from 950° C. to 1100° C., and especially from 975° C. to 1050° C. promote the formation of theta-alumina crystallites.

In some embodiments, the support has a surface area of 120 m²/g or less and a corresponding ABD of 0.50 g/cm³ or more. These characteristics are imparted in the alumina by a final calcination of the alumina at a temperature ranging from 950° C. to 1200° C. In some embodiments, the final calcination step is at conditions sufficient to convert the alumina into theta-alumina. Such conditions would include a calcination temperature closely controlled between 950° C. and 1100° C., and preferably from 975° C. to 1050° C.

The surface area of the catalyst as set forth is derived by the well-known mercury intrusion technique. This method may be used for determining the pore size distribution and pore surface area of porous substances by mercury intrusion using a Micromeritics Auto Pore 9200 Analyzer. In this method, high pressure mercury is forced into the pores of the catalyst particles at incrementally increasing pressures to a maximum of 413,700 kPa (60,000 psia). Pore volume readings are taken at predetermined pressures. A maximum of 85 pressure points can be chosen. Accordingly by this method, a thorough distribution of pore volumes may be determined.

The effect of calcination of an alumina base, especially at the elevated temperatures described here is to densify the alumina base. The densification, i.e. increase in ABD, is caused by a decrease in the overall catalyst pore volume. In addition, the high calcination temperatures cause the existing pores to expand. To accomplish this apparently contradictory mechanism, the catalyst necessarily contracts in size while the existing pores expand. By expanding, the mouths of the existing pores increase so that they become less likely to be plugged or restricted by coke build-up.

In some embodiments, the alumina component is essentially theta-alumina. By “essentially theta-alumina”, it is meant that at least 75% of the alumina crystallites are theta-alumina crystallites. The remaining crystallites of alumina will likely be in the form of alpha-alumina or gamma-alumina. However, other forms of alumina crystallites known in the art may also be present. The essentially theta-alumina component can comprise at least 90% crystallites of theta-alumina, if desired.

After the catalyst components have been combined with the support, the resulting catalyst composite will generally be dried at a temperature of from about 100° C. to about 320° C. for a period of typically about 1 to 24 hours or more and thereafter calcined at a temperature of about 320° C. to about 600° C. for a period of about 0.5 to about 10 or more hours. Typically, chlorine-containing compounds are added to air to prevent sintering of catalyst metal components. This final calcination typically does not affect the alumina crystallites or ABD. However, the high temperature calcination of the support may be accomplished at this point if desired. Finally, the calcined catalyst composite is typically subjected to a reduction step before use in the hydrocarbon conversion process. This reduction step is effected at a temperature of about 230° C. to about 650° C. for a period of about 0.5 to about 10 or more hours in a reducing environment, preferably dry hydrogen, the temperature and time being selected to be sufficient to reduce substantially all of the platinum group component to the elemental metallic state.

Suitable catalysts are described in U.S. Pat. Nos. 4,430,517, 4,914,075, and 6,756,340, each of which is incorporated herein by reference.

In one embodiment, the oxidative dehydrogenation catalyst comprises a platinum group component, a Group IVA component, an alkali or alkaline earth component, more than 0.2 weight %, calculated on an elemental basis, of a halogen component and a porous carrier material, wherein the atomic ratio of the alkali or alkaline earth component to the platinum group component is more than 10. The platinum group component is preferably present in the final composite in an amount, calculated on an elemental basis, of about 0.01 to 5 weight %; the Group IVA component is preferably present in an amount of about 0.01 to 5 weight %; the alkali or alkaline earth component is preferably present in an amount of about 0.01 to 15 weight %; and the halogen component is present preferably in an amount of about 0.2 to 15 weight %.

In another embodiment, the oxidative dehydrogenation catalyst comprises a first component selected from Group VIII noble metals, a second component selected from the group consisting of alkali or alkaline earth metals or mixtures thereof, and a third component selected from the group consisting of tin, germanium, lead, indium, gallium, thallium, or mixtures thereof, all on an alumina support having a surface area of 120 m²/g or less and an apparent bulk density of 0.5 g/cm³ or more.

In another embodiment, the oxidative dehydrogenation catalyst comprises a first component selected from Group VIII noble metal components or mixtures thereof, a second component in an amount from 0.9 to 1.1 weight percent, based on the total composite weight selected from the group consisting of alkali or alkaline earth metal components or mixtures thereof and a third component selected from the group consisting of tin, germanium, lead, indium, gallium, thallium and mixtures thereof, all on an alumina support comprising essentially theta-alumina and having a surface area from about 50 to about 120 m²/g and an apparent bulk density of at least 0.5 g/cm³ wherein the mole ratio of the first component to the third component is in the range from about 1.5 to about 1.7.

### EXAMPLE

Table 1 provides the operating conditions for a single-stage, and Table 2 compares the performance of the two systems. As the data illustrates, the single stage reactor system with oxidative preheat gives significantly higher selectivity.

TABLE 1

| | Single Stage Rxs |
|---|---|
| | with Oxidative Preheat |
| Charge Heater Temp, C. | 550 |
| O2 to Propane ratio, mol/mol | 0.057 |
| H2 to Propane Ratio, mol/mol | 0.44 |
| Steam to Propane Ratio, mol/mol | 5.6 |
| Reactor Inlet Pressure, psig | 10.7 |
| Dehydrogenation Temp after Oxidative | 650 |
| Preheat, C. | |
| Reactor Outlet Pressure, psig | 7.0 |
| Reactor Outlet Temp, C. | 540.5 |

TABLE 2

| | | Single-Stage Rxs |
|---|---|---|
| | Conventional Rxs | with Oxi-Preheat |
| Propane Conversion, % | 33.9 | 33.7 |
| Propylene Selectivity, wt % | 84.7 | 94.4 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A process for dehydrogenation of hydrocarbons comprising:
preheating a feed comprising a hydrocarbon feed and hydrogen to a temperature lower than a dehydrogenation temperature;
introducing an oxygen-containing gas to the preheated feed;
introducing the preheated feed with the oxygen-containing gas into a dehydrogenation reaction zone comprising a dehydrogenation reactor containing a dual functional catalyst having dehydrogenation and selective oxidation activities;
contacting the preheated feed with the catalyst in the dehydrogenation reactor to selectively oxidize the hydrogen to further heat the preheated feed; and
contacting the heated feed with the catalyst in the dehydrogenation reactor under dehydrogenation conditions to form olefins and hydrogen.

2. The process of claim 1 wherein the dehydrogenation zone comprises two or more dehydrogenation reactors connected in parallel, and wherein a portion of the preheated feed with the oxygen-containing gas is sent to each dehydrogenation reactor.

3. The process of claim 1 further comprising introducing a diluent before preheating the feed.

4. The process of claim 3 wherein the diluent comprises steam, methane, nitrogen, or combinations thereof.

5. The process of claim 1 further comprising introducing a diluent to the preheated feed before introducing the preheated feed with the oxygen-containing gas into a dehydrogenation reaction zone.

6. The process of claim 5 wherein the diluent comprises steam, methane, nitrogen, or combinations thereof.

7. The process of claim 1 wherein the hydrocarbon feed comprises C₂ to C₂₀ hydrocarbons.

8. The process of claim 1 wherein the feed is preheated to a temperature in a range of about 480 to about 580° C.

9. The process of claim 1 wherein after the oxidative heating, the heated feed is at a temperature in a range of about 500° C. to about 700° C.

10. The process of claim 1 wherein a selectivity to the olefins formed relative to an alkane dehydrogenated is increased compared to a selectivity to the olefins formed relative to an alkane dehydrogenated without the oxidative heating.

11. The process of claim 1 wherein the hydrocarbon feed comprises propane, the olefin comprises propylene, and wherein a selectivity to the propylene relative to the propane is at least 5 wt % higher than to a selectivity to the propylene relative to the propane without the oxidative heating.

12. The process of claim 1 further comprising separating unreacted hydrocarbons from the olefins, and recycling at least a portion of the unreacted hydrocarbons to the dehydrogenation reactor zone.

13. The process of claim 1 wherein a conversion of hydrocarbons to olefins is at least about 30%.

14. The process of claim 1 wherein the catalyst comprises a first component selected from the group consisting of Group VIII metal components and mixtures thereof supported on a support, a second component selected from the group consisting of alkali metal components, alkaline earth metal components, and mixtures thereof, and a third component selected from the group consisting of tin, germanium, lead, indium, gallium, thallium, and mixtures thereof.

15. A process for dehydrogenation of hydrocarbons comprising:
preheating a feed comprising a hydrocarbon feed and hydrogen to a temperature in a range of about 480 to about 580° C.;
introducing an oxygen-containing gas and steam to the preheated feed;
introducing the preheated feed with the oxygen-containing gas and steam into a dehydrogenation reaction zone comprising at least two dehydrogenation reactors connected in parallel, the at least two dehydrogenation reactors containing a dual functional catalyst having dehydrogenation and selective oxidation activities, a portion of the preheated feed with the oxygen-containing gas and steam being sent to each dehydrogenation reactor;
contacting the preheated feed with the catalyst in the at least two dehydrogenation reactors to selectively oxidize the hydrogen to further heat the preheated feed;
contacting the heated feed with the catalyst in the at least two dehydrogenation reactors under dehydrogenation conditions to form olefins and hydrogen.

16. The process of claim 15 further comprising introducing a diluent, before preheating the feed.

17. The process of claim 16 wherein the diluent comprises steam, methane, nitrogen, or combinations thereof.

18. The process of claim 15 wherein the hydrocarbon fed comprises C₂ to C₂₀ hydrocarbons.

19. The process of claim 1 wherein after the oxidative heating, the heated feed is at a temperature in a range of about 450° C. to about 700° C.

20. The process of claim 1 wherein a selectivity to the olefins formed relative to an alkane dehydrogenated is increased compared to a selectivity to the olefins formed relative to an alkane dehydrogenated without the oxidative heating, and wherein a conversion of hydrocarbons to olefins is at least about 30%.
